(19) United States
(12) Patent Application Publication
(10) Pub. No.: US 2013/0217930 A1
(43) Pub. Date: Aug. 22, 2013

(54) Use of Self-Crosslinked Siloxanes for the Defoaming of Liquid Hydrocarbons

(71) Applicant: Evonik Industries AG, (US)

(72) Inventors: Rene Haensel, Dorsten (DE); Michael Fiedel, Essen (DE); Michael Ferenz, Essen (DE); Joachim Venzmer, Essen (DE)

(73) Assignee: Evonik Industries AG, Essen (DE)

(21) Appl. No.: 13/771,973

(22) Filed: Feb. 20, 2013

(30) Foreign Application Priority Data
Feb. 20, 2012 (DE) 102012202523.5

(51) Int. Cl. C07C 7/20

(52) U.S. Cl. CPC C07C 7/20
USPC 585/3

(57) ABSTRACT

The present invention relates to the use of compositions comprising the following components: A, a polymer obtainable through hydrosilylation of a siloxane having SiH functions and having vinyl functions with another unsaturated compound, and D, platinum-group-metal atoms or platinum-group-metal ions, as antifoam for hydrocarbons, and also for suppressing or reducing the formation of foam or for the destabilization of foam.

The present application claims priority from German Patent Application No. DE 10 2012 202 523.5 filed on Feb. 20, 2012, the disclosure of which is incorporated herein by reference in its entirety.

### 1. FIELD OF THE INVENTION

The present invention relates to the use of compositions comprising the following components: A, a polymer obtainable through hydrosilylation of a siloxane having SiH functions and having vinyl functions with another unsaturated compound, and D, platinum-group-metal atoms or platinum-group-metal ions, as antifoam for hydrocarbons, and also for suppressing or reducing the formation of foam or for the destabilization of foam.

The present invention is directed to the use of a composition made of branched, organo-modified polysiloxanes having less than 25% by weight silicon content as antifoam for liquid hydrocarbons at high temperatures. The expression liquid hydrocarbons is also intended to include those organic systems that are composed predominantly, though not entirely, of organic compounds.

Examples of uses of liquid hydrocarbons for the purposes of the present invention are oils used in force-transmission applications, such as hydraulic oils, transmission oils or engine oils. Conveying, or pumped circulation, of these organic systems in the presence of air and sometimes of water can result in foaming, which can be severe. If sparingly soluble, polar organic compounds are present this foam is often stable, since the sparingly soluble, polar compounds can act as stabilizers at the interface in the air-in-oil dispersion that is the foam. Foaming can also occur at high temperatures in these applications, for example at >100° C. Foaming occurs on the surface of the organic system during escape of gas, and must be suppressed, since foam alters the rheological and chemical properties of the systems, for example through oxidation, and by way of example can have an adverse effect on lubrication and force-transmission processes and thus on the lifetimes of oil and of machinery.

Other liquid hydrocarbons for the purposes of the present invention are those arising during the distillative work-up of crude oil at atmospheric pressure, and during the vacuum distillation process. Some of the distillation processes are carried out by way of example in refineries at high temperatures. In these processes, foams reduce the quality of separation and are detrimental to the capacity of the separation apparatus. Cracking processes in refineries moreover often involve severe foaming under extreme temperature conditions, for example during operation of what are known as visbreakers or cokers. Foams reduce the cost-effectiveness of these entire processes.

It is noted that citation or identification of any document in this application is not an admission that such document is available as prior art to the present invention.

Other processes carried out at room temperature, and also at temperatures below 100° C., use polyalkylsiloxanes as antifoams, preferably polydimethylsiloxanes, often abbreviated to PDMS in the literature, and known as silicone oils. These are incompatible materials, frequently resulting in separation and thus reduced long-term effect, and they have high silicon content, which is often undesirable, and for these reasons PDMSs are substantially being replaced by organo-modified siloxanes under the said conditions.

Examples of materials that are effective as antifoams at temperatures below 100° C. are conventional silicone-polyether copolymers. These comprise one or more polyethers, produced through an addition reaction of monomers, such as propylene oxide and/or ethylene oxide, onto starter alcohols, such as allyl alcohol. These products are effective even if the amount added is as little as 2 ppm.

Silicone-polyether copolymers are effective because they have only partial solubility in the medium requiring defoaming, and by virtue of their surfactant effect they migrate to the interface. The structure of the silicone-polyether copolymers is modified specifically to be appropriate to the prevailing conditions. It is essential here that the surface tension of the antifoam is lower than that of the liquid requiring defoaming, so that the antifoam can migrate to the oil-air interface, where its spreading properties lead to destruction of the foam bubbles. This known mechanism is described by Denkov D.N. in “Mechanisms of Action of Mixed Solid-Liquid Antifoams—Stability of Oil Bridges in Foam Films”, Langmuir, 1999. The requirement for partial insolubility in the hydrocarbons requiring defoaming here, together with lower surface tension, can be a reason for the restricted use of purely organically based antifoams for these applications in industry: they are substantially less effective, because purely organic antifoams do not generally have these two properties simultaneously.

The general structure in DD 213945 A serves as an example for the defoaming of hydrocarbons at low temperatures. Foam inhibitors for mineral-oil-based lubricating oils are proposed here. The compounds proposed are based on a polysiloxane structure with pendant polyether moieties terminated by butyl groups.

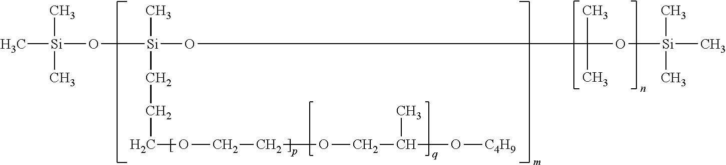

DE 4343235 C (U.S. Pat. No. 5,613,988) describes another example of organo-modified siloxanes for the defoaming of hydrocarbons, e.g. diesel fuels, at room temperature. That publication describes a process for the defoaming of diesel fuel by using organo-functional polysiloxanes of the general formula

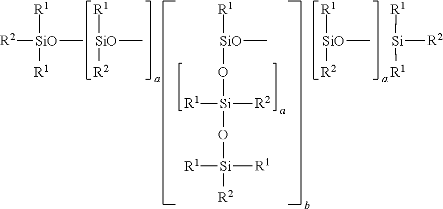

where the radicals R¹ are alkyl or aryl radicals, and the radicals R² are selected from a plurality of the following classes: butene derivatives, alkanol derivatives, polyethers and alkyl radicals.

U.S. Pat. No. 5,542,960 and U.S. Pat. No. 5,334,227 describe the use of organopolysiloxanes for the defoaming of diesel fuel. Here, polysiloxane terpolymers used as diesel antifoams have the structure MDₓD*_{y}D**_{z}M, where M=O_{0.5}Si(CN₃)₃, D=OSi(CH₃)₂, D*=OSi(CH₃)R, where R=a polyether, D**=OSi(CH₃)R′, where R′=phenol derivative and x+y+z=from 35 to 350, x/(y+z)=from 3 to 6 and y/z=0.25 to about 9.0. The organopolysiloxanes described in U.S. Pat. No. 5,334,227 comprise polyethers which are composed of more than 75% of ethylene oxide units and which have no butylene oxide units.

The conventional organopolysiloxanes described above become ineffective or have only significantly poorer effectiveness in hydrocarbon systems requiring defoaming at relatively high temperatures above 100° C., as can be the case with engine oils or with distillation processes, e.g. in refineries, and in cracking processes. A possible reason for this is that the temperature increase leads to better compatibility with the hydrocarbons requiring defoaming. At even higher temperatures, the products probably undergo thermal degradation, and the degradation products are probably then ineffective because of low molecular weight.

Other methods for the defoaming of hydrocarbons at relatively high temperatures for the various applications therefore uses high-molecular-weight, high-viscosity polydimethylsiloxanes, for example the Dow Corning product group including silicone oils with various viscosities marketed as “Dow Corning 200 Fluid”. Silicone oils are effective even at low concentrations starting at 2 ppm. However, they have the disadvantage already described: the high silicon content, about 38% by weight in the case of the polydimethylsiloxanes mainly used, is undesirable for many applications. In refineries, silicon-containing degradation products lead to poisoning of catalysts which are by way of example used in downstream processes, e.g. the hydrotreatment process. Another disadvantage of silicone oils is that they have an adverse effect on air-release properties relating to air dispersions, known as microfoam, within the oil. This is a disadvantage for the use of oils as lubricants, for example in transition systems and hydraulic systems, since dispersed air adversely affects the tribological properties of the lubricant. The air bubbles dispersed within the material lead to a lubrication film which is inhomogeneous and therefore unstable and which by way of example is more susceptible to break-out or separation between tooth faces. The theoretical principles of the mechanism of formation of air dispersions in oil and of surface foam derived from dissolved and mechanically incorporated air, and the possible consequences for industrial operations, are known (Tourret & White: “Aeration and Foaming in Lubricating Oil Systems”; Aircraft Engng. 24 (1952), 122-130, 137). It is therefore known that the requirements for inhibiting surface foam and air dispersion are contradictory. Surfactant substances (e.g. silicone oils) which inhibit the production of foam have an adverse effect on air-release properties (ARP). The organo-modified siloxanes of DD-A-213 945 are known to impair air-release properties less than the silicone oils, but they are useful only at low temperatures.

US20050109675 moreover describes the defoaming of non-aqueous systems. The addition of a silicone resin to a linear polydimethylsiloxane increases the effectiveness of the linear silicone oil. However, thermal degradation of the linear silicone oil leads to volatile cracking products and to decreasing effectiveness at high temperatures.

U.S. Pat. No. 4,082,690 moreover describes mixtures made of polydimethylsiloxanes with silicone resins, known as MQ resins, in a hydrocarbon for the defoaming of non-aqueous systems. However, these silicone-containing antifoams have the disadvantage of high Si content: about 38% by weight.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

It is further noted that the invention does not intend to encompass within the scope of the invention any previously disclosed product, process of making the product or method of using the product, which meets the written description and enablement requirements of the USPTO (35 U.S.C. 112, first paragraph) or the FPO (Article 83 of the EPC), such that applicant(s) reserve the right to disclaim, and hereby disclose a disclaimer of any previously described product, method of making the product, or process of using the product.

### SUMMARY OF THE INVENTION

An object of the present invention is therefore the use of compositions based on organo-modified siloxanes which are suitable as antifoams which do not exhibit one or more of the disadvantages of the siloxane-based antifoams described above, in particular exhibiting markedly less contamination of the products of a thermal process by low-molecular weight silicon-containing decomposition products.

Surprisingly, it has been found that compositions comprising components A and D and optionally B and/or C, as defined hereinafter, achieve the said object.

The present invention therefore provides the use of compositions comprising components A and D and optionally B and/or C as described in the claims.

An advantage of the use according to the invention is that, after use of thermal processes, the products have a markedly reduced silicon content when comparison is made with the known defoaming methods.

Another advantage of the use according to the invention is that the compositions comprising components A and D and optionally B and/or C can be used over a very wide temperature range.

### BRIEF DESCRIPTION OF THE DRAWINGS

FIG. 1 is a diagram of an apparatus for carrying out defoaming experiments in what is known as the frit test.

### DETAILED DESCRIPTION OF EMBODIMENTS

It is to be understood that the figures and descriptions of the present invention have been simplified to illustrate elements that are relevant for a clear understanding of the present invention, while eliminating, for purposes of clarity, many other elements which are conventional in this art. Those of ordinary skill in the art will recognize that other elements are desirable for implementing the present invention. However, because such elements are well known in the art, and because they do not facilitate a better understanding of the present invention, a discussion of such elements is not provided herein.

The present invention will now be described in detail on the basis of exemplary embodiments.

The use of compositions is described hereinafter by way of example, without any intention that the invention be restricted to these examples of embodiments. Where ranges, general formulae or classes of compounds are mentioned hereinafter, these are intended to comprise not only the corresponding ranges or groups of compounds explicitly mentioned but also all of the subranges and subgroups of compounds that can be obtained by extracting individual values (ranges) or compounds. When documents are cited for the purposes of the present description, the entire content of these is intended to be part of the disclosure of the present invention. When content data (ppm or %) is provided hereinafter or hereinbefore, unless otherwise stated this involves data in % by weight or ppm by weight. The content data for compositions is based on the entire composition unless otherwise stated. When average values are provided hereinafter, unless otherwise stated these are numeric averages. When molar masses are used, unless expressly otherwise stated these are weight-average molar masses Mw. When measured values are provided hereinafter, unless otherwise stated the said measured values were determined at a pressure of 1013.25 hPa and at a temperature of 23° C.

The definitions hereinafter may contain other expressions used as equivalents and synonyms for the defined expression.

Unless otherwise stated, for the purposes of the invention the expression “molecular weight” means the mass-average molar mass, also termed average molecular weight, abbreviated to Mw and used with the unit g/mol.

The prefix “poly” is used in the context of this invention not merely exclusively for compounds having at least 3 repeating units of one or more monomers within the molecule, but also in particular for compositions of compounds which have a molecular weight distribution and thereby have an average molecular weight of at least 200 g/mol. This definition takes account of the fact that in the technical sector under consideration it is conventional to use the term polymers for compounds of this type even if they do not appear to comply with a definition of polymer on the basis of OECD or REACH guidelines.

“Liquid under conditions of usage” means that the medium requiring defoaming is liquid under the selected prevailing conditions of a process, which may be a thermal process. To this end it can sometimes be necessary to melt the medium, and this means that it is solid under standard conditions, for example 25° C. and 1013 mbar, and then becomes liquid on heating. The term liquid means, for these purposes, that the viscosity of the medium is below 120 Pa*s, preferably up to or below 100 Pa*s. The liquid medium can optionally also comprise solids.

The indices a, b, c, d, e, f, g, h, j, k, l, m, n, o, p, q and r given here, and also the value ranges of the indices given, can be understood to be average values of the possible statistical distribution of the actual structures present and/or mixtures of these. This also applies to structural formulae reproduced as such in a form which is per se precise, an example being formula (I), (II), (III) or (IV).

A feature of the use according to the invention is that compositions are used for the defoaming of hydrocarbons that are liquid under conditions of usage, where the compositions according to the use comprise components A and D, where A comprises a polymer obtainable through a hydrosilylation reaction of compounds of the formula (I)

MₐM^{v}_{b}M^{H}_{c}D_{d}D^{H}ₑD^{v}_{f}T_{g}Qₕ formula (I)
where
   M=[R¹₃SiO_{1/2}]
   M^{V}=[R³R¹₂SiO_{1/2}]
   M^{H}=[R¹₂SiHO_{1/2}]
   D=[R¹₂SiO_{2/2}]
   D^{H}=[R¹SiHO_{2/2}]
   D^{V}=[R³R¹SiO_{2/2}]
   T=[R¹SiO_{3/2}]
   Q=[SiO_{4/2}]
   a=from 0 to 42, preferably from 0 to 22, particularly preferably from greater than 0 to 2,
   b=from 0 to 42, preferably from greater than or equal to 1 to 22, particularly preferably from greater than 1 to less than 2,
   c=from 0 to 42, preferably from 0 to 22, particularly preferably 0,
   d=from 5 to 600, preferably from 10 to 400, more preferably from 20 to 300, particularly preferably from 50 to 200,
   e=from 0 to 50, preferably from greater than 0 to 25, more preferably from 0.5 to 10, particularly preferably from 0.7 to 1.5,
   f=from 0 to 50, preferably from 0 to 25, particularly preferably 0,
   g=from 0 to 20, preferably from greater than 0 to 10, particularly preferably from 1 to 5,
   h=from 0 to 20, preferably from 0 to 10, particularly preferably 0,
with the proviso that the following conditions are satisfied:
   a+b+c is greater than or equal to 2,
   b+f is greater than 0, preferably greater than or equal to 1.2 and smaller than 2,
   c+e is greater than 0, preferably greater than 0.8, more preferably from greater than 1 to 8, particularly preferably from 1.2 to less than 6
   and 0.24*(a+b+c+d+e+f+g) is greater than (c+e),
R1 are mutually independently identical or different alkyl radicals having from 1 to 30 carbon atoms, or identical or different aryl radicals having from 6 to 30 carbon atoms or —OH or —OR2,
   preferably methyl, phenyl, —OH or —OR²,
   particularly methyl or phenyl,
R² are mutually independently identical or different alkyl radicals having from 1 to 12 carbon atoms, or identical or different aryl radicals having from 6 to 12 carbon atoms,
   preferably methyl, ethyl, n-propyl, isopropyl, n-butyl, sec-butyl, isobutyl, phenyl,
   particularly methyl and ethyl,
R³ are mutually independently identical or different organic radicals having a terminal double bond or having a terminal or internal triple bond, preferably organic radicals having a terminal double bond,
   particularly vinyl (i.e. —CH═CH2) or allyl (i.e. —CH2CH═CH2),
with compounds of the formula (I) and/or with other compounds C which have a C-C multiple bond and do not correspond to formula (I),
   and
   D platinum-group-metal atoms and/or platinum-group-metal ions, preferably platinum atoms, rhodium atoms and ruthenium atoms, in particular platinum atoms.

The compounds of the formula (I) can be termed self-crosslinking siloxanes. They are characterized in that they have, alongside SiH functions, multiple bonds accessible to hydrosilylation, and therefore in that two or more compounds of the formula (I) can react with one another in a hydrosilylation reaction.

Compositions that can be used according to the invention are those which comprise component A exclusively or comprise, alongside other polymers, a polymer which is obtainable through a hydrosilylation reaction of compounds of the formula (I) and compounds of the formula (II)

MᵢM^{H}ᵢDₖD^{H}ₗTₘQₙ formula (II)
where
   i=from 0 to 34, preferably from 0 to 18, particularly preferably from greater than 0 to 2,
   j=from 0 to 34, preferably from 0 to 18, particularly preferably from greater than 0 to 2,
   k=from 5 to 600, preferably from 10 to 400, more preferably from 20 to 200, particularly preferably from 50 to 150,
   l=from 0 to 50, preferably from greater than 0 to 35, more preferably from 1 to 26, particularly preferably from greater than 1 to 10,
   m=from 0 to 16, preferably from 0 to 8, particularly 0,
   n=from 0 to 16, preferably from 0 to 8, particularly 0,
and with the following conditions:
   i+j is greater than or equal to 2 and
   j+l is greater than or equal to 2.

It is preferable that the compositions according to the use comprise a component A which comprises a polymer obtainable through a hydrosilylation reaction of compounds of the formula (I) with one or more unsaturated compounds C. It is preferable that the compositions according to the invention comprise a component A which comprises a polymer obtainable through a hydrosilylation reaction of compounds of the formula (I) with a compound of the formula (II) and with one or more unsaturated compounds C.

It can be advantageous for the compositions according to the use to comprise a component B obtainable through a hydrosilylation reaction of compounds of the formula (II) as defined above and unsaturated compounds C.

The compositions according to the use can comprise one or more compounds C, and these can be added subsequently to the composition or can remain in the form of unreacted reactant in the composition, during production of the composition.

The abovementioned compounds C are preferably olefins or polyethers which have one or more carbon-carbon multiple bonds, preferably polyethers which have one or more carbon-carbon multiple bonds.

Preferred olefins are olefins having terminal double bonds, e.g. alpha-olefins, alpha-omega-olefins, mono- and polyols containing allyl groups or aromatics containing allyl groups. Particularly preferred olefins are ethene, ethyne, propene, 1-butene, 1-hexene, 1-dodecene, 1-hexadecene, 1,3-butadiene, 1,7-octadiene, 1,9-decadiene, styrene, eugenol, allylphenol, methyl undecylenate, allyl alcohol, allyloxyethanol, 1-hexen-5-ol, allylamine, propagyl alcohol, propagyl chloride, propagylamine or 1,4-butynediol.

Examples of preferred polyethers having one or more multiple bonds are allyl-functional polyethers or 1,4-butynediol-started polyethers. Particularly preferred polyethers which have carbon-carbon multiple bonds are preferably those of the formula (III),

CH₂═CHCH₂O(C₂H₄O)ₒ(C₂H₃(CH₃)O)ₚ(C₂H₃(C₂H₅)O)_{q}(C₂H₃(Ph)O)ᵣR⁴ formula (III)

where
R⁴ are mutually independently identical or different organic radicals which do not contain any multiple bond accessible to hydrosilylation,
   preferably hydrogen, alkyl radicals or carboxy radicals,
   particularly preferably hydrogen, methyl, butyl or acetyl,
   particularly preferably hydrogen,
o=from 0 to 200, preferably from greater than 0 to 150, particularly preferably from greater than or equal to 2 to 120, particularly preferably from greater than or equal to 3 to 90,
p=from 0 to 200, preferably from greater than 0 to 150, particularly preferably from greater than or equal to 2 to 100, particularly preferably from greater than or equal to 3 to 80,
q=0 or from greater than 0 to 100, preferably 0 or from greater than 0 to 30, particularly preferably 0 or from greater than 0 to 1, particularly 0,
r=0 or from greater than 0 to 100, preferably 0 or from greater than 0 to 30, particularly preferably 0 or from greater than 0 to 1, particularly 0,

and with the following conditions:
o+p+q+r is greater than 3, and preferably o is greater than 0, more preferably o is greater than p+q+r; o is particularly preferably greater than p, and with particular preference o is greater than 1.5*p, and
if q+r is less than 2, o is then greater than 4*p,
in the event that p=0, o is at least 4, preferably at least 8,
in the event that p=0 and q+r is equal to or greater than 2, o is at least 2*(q+r).

Examples of very particularly preferred polyethers are:
CH₂═CHCH₂O(C₂H₄O)₂₀(C₂H₃(CH₃)O)_{4.5}H
CH₂═CHCH₂O(C₂H₄O)₂₅(C₂H₃(CH₃)O)_{4.5}H
CH₂═CHCH₂O(C₂H₄O)₈H
CH₂═CHCH₂O(C₂H₄O)₂₀(C₂H₃(CH₃)(O)_{4.5}Me
CH₂═CHCH₂O(C₂H₄O)₂₆(C₂H₃(CH₃)O)_{4.5}Me
CH₂═CHCH₂O(C₂H₄O)₈Me
CH₂═CHCH₂O(C₂H₄O)₂₀(C₂H₃(CH₃)O)_{4.5} acetyl
CH₂═CHCH₂O(C₂H₄O)₂₆(C₂H₃(CH₃)O)_{4.5} acetyl
CH₂═CHCH₂O(C₂H₄O)₈ acetyl
CH₂═CHCH₂O(C₂H₄O)₈(C₂H₃(Ph)O)₃H
CH₂═CHCH₂O(C₂H₄O)₈(C₂H₃(Ph)O)₂H

Polyethers having one or more carbon-carbon multiple bonds are available commercially, e.g. with the trade mark Pluriol® (BASF) or Polyglycol AM® (Clariant).

Particular preference is given to use according to the invention of compositions comprising component A which itself comprises compounds of the formula (I) where
a=from 0 to 2,
b=from greater than 1 to 22,
c=from 0 to 22,
d=from 50 to 400,
e=from 0 to 1.5,
f, g, h=from 0 and the definitions of the radicals are as given above,
   and compounds of the formula (H), where
i=from 1 to 2,
j=from 0 to 1,
k=from 50 to 150,
l=greater than 1 to 26,
   component B,
   compounds C of the formula (III), where
if q+r is less than 2, o is then greater than 4*p,
in the event that p=0, o is at least 8,
in the event that p=0 and q+r is equal to or greater than 2, o is at least 2*(q+r),
   and component D, taking the form of platinum atoms and/or platinum ions.

Although this theory is not necessarily correct it is believed that when the compositions according to the use are used as antifoams in hydrophobic systems their activity derives from their lack of miscibility with the medium requiring defoaming (ref. Venzmer et al. in Bartz, W. J. (Ed.): 5th International Colloquium Fuels, TAE Esslingen, 12-13 Jan. 2005, 483-488). Hydrophilic substituents in components A, B and D, and also in compound C, for example polyethylene-rich molecular radicals, polyethylene-glycol-like molecular radicals and/or hydroxyl groups, can therefore be particularly useful here and are therefore preferred.

It is preferable that the compositions according to the use comprise
a proportion of 1 to 90% by weight, preferably from greater than 1 to 30% by weight, with preference from 1 to 15% by weight, of component A,
a proportion of from 0 to 70% by weight, preferably from greater than 0 to 40% by weight, with preference from 1 to 30% by weight, of component B,
a proportion of from 0 to 95% by weight, preferably from 5 to 90% by weight, with preference from 10 to less than 90% by weight, of the compounds C and
a proportion of from greater than 0 to 50 ppm by weight of component D,
based in each case on the mass of the entire composition.

The compositions according to the use comprise with preference
a proportion of from 1 to 15% by weight of component A,
a proportion of from 1 to 30% by weight of component B,
a proportion of from 10 to less than 90% by weight of the compounds C and
a proportion of from greater than 0 to 50 ppm by weight of component D,
based in each case on the mass of the entire composition.

In the compositions according to the use, more than 90% by weight of the polymer of component A, based on components A, has a weight-average molar mass of less than 2 500 000 g/mol.

In the compositions according to the use, more than 90% by weight of component B, based on component B, has a weight-average molar mass of up to 1 000 000 g/mol. The amount of this type of component B in the composition is preferably less than 5% by weight, based on the entire composition.

A feature of preferred compositions according to the use is that component A comprises, based on components A, more than 90% by weight of polymers with a weight-average molar mass of less than 2 500 000 g/mol and more than 90% by weight of component B, based on component B, has a weight-average molar mass of up to 1 000 000 g/mol, and the amount of component B present in the composition is less than 5% by weight, based on the entire composition.

The compositions of the use according to the invention are preferably liquid at 20° C. and 1013 mbar. For the purposes of the invention, substances and homogeneous and/or heterogeneous mixtures are liquid if their viscosity at room temperature, preferably at 20° C. and atmospheric pressure (1013 mbar) is smaller than 120 Pa*s, preferably smaller than 100 Pa*s and particularly preferably smaller than 10 Pa*s. Accordingly, preferred compositions have an appropriate viscosity, determined as stated in the examples.

A feature of the compositions of the use according to the invention is preferably that their content of silicon, based on the total of the masses of components A, B and D and compound C of the composition, is less than 25% by weight, with preference less than 20% by weight, with particular preference less than 15%, and with very particular preference from 0.01 to 10% by weight.

The content of platinum-group-metal atoms and/or platinum-group-metal ions in the compositions of the use according to the invention is preferably from greater than 0 to 50 wppm (ppm by mass), with preference from 1 to 40 wppm, with particular preference from 3 to 30 wppm, with very particular preference from 5 to 20 wppm and with preference in particular from 8 to 10 wppm, based on the total mass of the composition. It is preferable that platinum, ruthenium and/or rhodium is present at the said concentrations in the composition.

The compositions according to the use can optionally comprise further additions. Preferred additions are aliphatic and/or aromatic oils and solvents.

Examples of preferred solvents are alcohols and aliphatic hydrocarbons. Preferred alcohols can by way of example be methanol, ethanol, ethylene glycol, n-propanol, isopropanol, propylene 1,2-glycol, propylene 1,3-glycol, n-butanol, 2-butanol and tert-butanol. Preferred hydrocarbons are particularly hydrocarbons with a boiling point below 250° C. at atmospheric pressure (1013 mbar).

The compositions of the use according to the invention optionally comprise compounds characterized by the partial structure of the formula (V).

CH₃—CH═CH₂— formula (V)

Preferred compounds comprising the partial structure of the formula (V) are polyethers of the formula (IV)

CH₃—CH═CH₂—O(C₂H₄O)ₒ(C₂H₃(CH₃)O)ₚ(C₂H₃(C₂H₅)O)_{q}(C₂H₃(Ph)O)ᵣR⁴ formula (IV)

where the definitions of the indices and of the radical R⁴ are as for formula (III). The preferred ranges stated above for formula (III) also apply in identical fashion for the compounds of the formula (IV).

The compounds of the formulae (IV) and/or (V) can be added additionally to the composition according to the use or by way of example can be produced by rearrangements at C-C multiple bonds during the course of the production of the composition, particularly during the reaction under hydrosilylating conditions.

The proportion of compounds having a partial structure of the formula (V), preferably compounds of the formula (IV), based on the composition according to the invention, is preferably from 0.0001 to 25% by weight, with preference from 0.01 to 20% by weight.

It can be advantageous for the composition according to the use to comprise no compounds having a partial structure of the formula (V), or for the proportion to be too small to be detectable by analysis.

The distribution of the various fragments in the formulae (I), (II), (III) and (IV) can be random. Random distributions can have a block structure with any desired number of blocks and with any desired sequence, or they can be subject to a randomized distribution, and can also have alternating structure or else form a gradient along the chain, and in particular they can also form any of the hybrids where groups of different distributions can optionally follow one another.

The compositions of the use according to the invention can be obtained in a manner known per se through a hydrosilylation reaction. Suitable catalysts for the hydrosilylation reaction are in principle platinum compounds, for example hexachloroplatinic acid, cisplatin, bis(cyclooctene)platinum dichloride, carboplatin, divinyltetramethyldisiloxane complexes of platinum(0), the substances known as Karstedt catalysts, or else various olefin complexes of platinum(0). Other substances suitable in principle are rhodium compounds, iridium compounds and ruthenium compounds, for example tris(triphenylphosphine)rhodium(I) chloride or tris(triphenylphosphine)ruthenium(II) dichloride. Catalysts preferred for the purposes of the process are complexes of platinum(0), and particular preference is given to Karstedt catalysts or the substances known as WK catalysts, produced in accordance with EP1520870.

During the course of a hydrosilylation reaction, a number of side reactions can occur. By way of example, the person skilled in the art is aware that SiOC bonds can be formed through the dehydrogenative addition reaction of alcohol functions onto the SiH bond. A small proportion of residual water can also lead to the degradation of SiH functions. Rearrangements at C-C multiple bonds are also known.

The compositions are preferably used for the defoaming of diesel fuels, of lubricating oils in transmission systems, engines or hydraulic systems, or else in thermal processes. The thermal processes can maintain the structural identity of the component(s) requiring defoaming or else alter its identity. These alterations can be of destructive type, e.g. involving decomposition and/or of constructive type, e.g. leading to the formation of new or desired substances. Examples of preferred thermal processes are distillation processes or cracking processes. The thermal processes can be carried out either at atmospheric pressure or else at subatmospheric pressure (under vacuum) or superatmospheric pressure. Particularly preferred uses are found in distillation processes, either in vacuo or else at atmospheric pressure. Preference is further given to applications in cracking processes.

The compositions can advantageously be used at temperatures of from 80 to 200° C., preferably from 100° C. to 180° C., particularly preferably from 120 to 160° C. These temperatures are preferably used in distillation processes.

The compositions can moreover advantageously be used at temperatures of up to 600° C., preferably from 150 to 500° C., more preferably from 200 to 450° C., still more preferably from 250 to 400° C., particularly from 300 to 350° C. These temperatures are preferably used in cracking processes. Cracking processes can by way of example be carried out in delayed cokers or visbreakers.

It is preferable that the compositions are used in distillation processes and/or cracking processes in refineries.

The compositions can moreover advantageously be used for the defoaming of hydrocarbons that are liquid under conditions of usage, for example lubricants and oils used in force-transmission applications. The temperatures here can be markedly above 100° C.

The compositions can be added to the hydrocarbons requiring defoaming directly, i.e. without other additions, or else in a mixture with aliphatic or aromatic solvents. It is preferable that the compositions according to the invention are used in a mixture with aliphatic or aromatic solvents or mixtures of these.

For purposes of the use according to the invention, the compositions can be added continuously or in batches to the hydrocarbons requiring defoaming. In the case of distillation processes, the compositions are preferably added continuously. The addition of the compositions can also be determined by the use of sensors to determine the amount of foam or its height. If the height of the foam exceeds a certain limiting value, addition of the antifoam is triggered. This addition can take place either into the liquid phase of the hydrocarbon requiring defoaming or else directly onto the foam. In the case of addition onto the foam, there are various possible distributive addition methods, preference being given to a dispersion in the ambient atmosphere, particularly preferably using a spray method. Particular preference is given to addition of the compositions onto the foam by spraying in cracking processes.

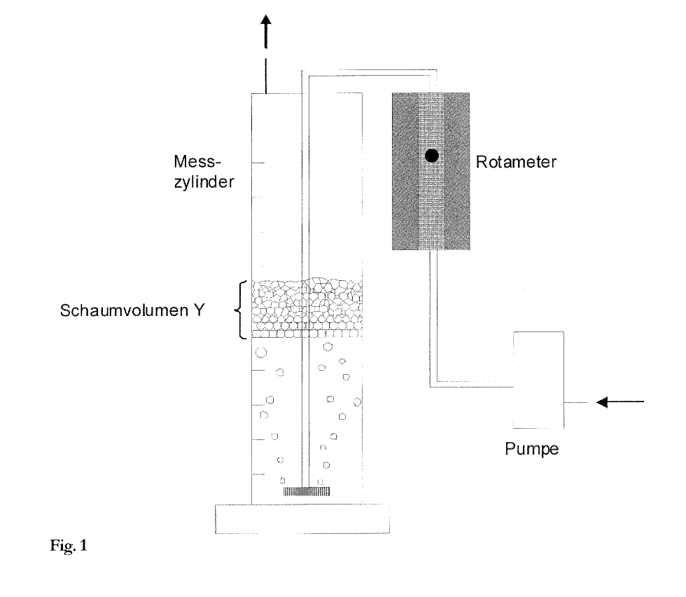

The present invention is explained in more detail with reference to FIG. 1, but there is no intention to restrict the invention, the range of usage of which is apparent from the entire description and from the claims, to the embodiment specified in the FIGURE. FIG. 1 is a diagram of an apparatus for carrying out defoaming experiments in what is known as the frit test.

The present invention is described by way of example in the examples listed hereinafter, but there is no intention to restrict the invention, the range of usage of which is apparent from the entire description and from the claims, to the embodiments specified in the examples.

### EXAMPLES

### General Methods and Materials

Viscosity—Viscosity Determination by Means of a Brookfield LV-DV-I+ Spindle Viscometer

Brookfield viscometers are rotary viscometers with defined spindle sets as rotors. The rotors used were an LV spindle set. Because viscosity is temperature-dependent, the temperatures of viscometer and of liquid requiring measurement were maintained precisely constant at 20° C.+/−0.5° C. during measurement. Other materials used alongside the LV spindle set were a thermostatic water bath, a 0-100° C. thermometer (scale divisions of 1° C. or smaller) and a chronometer (scale values not greater than 0.1 second). The measurement was carried out by charging 100 ml of the sample to a wide-necked flask, and carrying out measurements at controlled temperature and in the absence of air bubbles, after prior calibration. The positioning of the viscometer in relation to the sample for viscosity determination was such that the spindle is immersed up to the mark in the material. The measurement is triggered by the start button, and care was taken here that the measurement took place within the most advantageous measurement range of 50% (+/−20%) of the maximum measurable torque. The measurement result was displayed in mPas on the viscometer display, whereupon division by the density (g/ml) gives the viscosity in mm²/s.

Determination of SiH Content

The SiH values of the hydrosiloxanes used, and also those of the reaction matrices, are determined respectively by a gas-volumetric method by using the sodium-butoxide(butylate)-induced decomposition of sample aliquots, in a gas burette system. When the measured volumes of hydrogen are inserted into the ideal gas equation they enable determination of the content of active SiH functions in the starting materials, and also in the reaction mixtures, and thus permit control of the reaction.

Determination of Molar Mass Distributions:

Hewlett-Packard 1100 equipment was used for the gel permeation chromatography analyses (GPC), using an SDV column combination (1000/10 000 Å, in each case 65 cm, internal diameter 0.8 cm, temperature 30° C.), THF as mobile phase, with flow rate 1 ml/min and an RI detector (Hewlett-Packard). The system was calibrated against a polystyrene standard in the range from 162 to 2 520 000 g/mol.

The GPC analyses were carried out at various temperatures. The respective measured values were taken from the linear region of the measurements, and the molar masses were calculated against the selected standard.

Materials:

| Material | | Supplier |
|---|---|---|
| Decamethylcyclopenta- | D₅ | ABCR (cat. no. |
| siloxane | | AB111012) |
| Trifluoromethanesulfonic | TFMSA | Aldrich (cat. no. |
| acid | | 347817) |
| Solvesso 150 (aliphatic | | Exxon Mobil |
| solvent) | | Corporation |
| Divinyltetramethyldi- | | ABCR (cat no. |
| siloxane | | AB121873) |
| Polymethylphenylsiloxane | | ABCR, item no. |
| (500 cSt) | | PMM-0025 |
| Allylpolyether 1 | Iodine number = | |
| | 18.5 g/100 g, OHN = | |
| | 44 mg KOH/g, | |
| | 23% by weight of PO | |

The Karstedt solutions used are divinyltetramethyldisiloxane complexes of platinum(0) in decamethylcyclopentasiloxane at a concentration of 0.1% by weight of platinum (obtainable from Umicore with 21.37% by weight of platinum, adjusted to 0.1% by weight of Pt by dilution with decamethylcyclopentasiloxane). The amounts added of the catalyst as stated in the examples below are based on the total mass of the input weights of the components for the hydrosilylation reaction, added solvents being ignored in this calculation.

### Example 1

### SYNTHESIS EXAMPLES

### S1 (Starting Material for Synthesis Example S3)

### Production of an Equilibrate According to Formula (I)

3.1 g of divinyltetramethyldisiloxane, 0.98 g of a multiple-centrally-positioned hydrosiloxane (15.7 eq/kg of SiH), 194.7 g of D₅ and 0.12 ml of trifluoromethanesulfonic acid are used as initial charge in a multi-necked flask equipped with stirrer apparatus, nitrogen supply and reflux condenser, and are stirred for 24 hours at room temperature. After complete equilibration, the mixture is neutralized by addition of 4.0 g of sodium hydrogencarbonate within 2 hours, and then filtered. A colourless, clear silicone equilibrate was obtained.

### S2: Production of an Equilibrate According to Formula (II), R¹: Me and Phenyl)

13.33 g of a multiple-centrally-positioned hydrosiloxane (15.7 eq/kg of SiH), 65.05 g of D₅, 21.6 g of a polymethylphenylsiloxane (500 cSt, ABCR) and 0.1 ml of trifluoromethanesulfonic acid are used as initial charge in a multi-necked flask equipped with stirrer apparatus, nitrogen supply and reflux condenser, and are stirred for 24 hours at room temperature. After complete equilibration, the mixture is neutralized by addition of 6.0 g of sodium hydrogencarbonate within 2 hours, and then filtered. A colourless, clear silicone equilibrate was obtained.

### S3

14 g of a siloxane of the formula M₁M^{H}₁D₁₂₃D^{H}₂₅T₀Q₀ (R¹=Me) are mixed intimately with 69.94 g of allyl polyether 1, 2.98 g of an M_{0.04}M^{v}_{1.96}M^{H}₀D_{147.1}D^{H}_{0.9}D^{v}₀T₀Q₀ siloxane (S1), and also 20 g of Solvesso 150 in a multi-necked flask with blade stirrer with precision glass gland, reflux condenser, inert gas supply and thermometer, and the hydrosilylation reaction was initiated in an inert gas atmosphere by addition of 10 ppm of platinum in the form of a Karstedt catalyst to the cloudy reaction mixture. The mixture was then heated to a reaction temperature of from 80 to 90° C., and the exothermic reaction was controlled so as to avoid exceeding a reaction temperature of 90° C. After 2.5 hours it was not possible to detect any residual free SiH, and conversion was complete. The molar mass of the yellowish product according to GPC analysis was Mₙ=4744 g/mol and M_{w}=164457 (M_{w}/Mₙ=34.67) and its viscosity was 3.1 Pa s. The Si content of the resultant product was ˜8% by weight, based on the pure substance, i.e. without solvent.

### S4

16.1 g of a siloxane of the formula M₂M^{H}₀D₆₇D^{H}₂₄T₀Q₀ (where R¹=Me or phenyl) which was produced in S2 are intimately mixed with 59.4 g of allyl polyether 1, 4.4 g of an M₀M^{v}₂M^{H}₀D₃₅₀D^{H}₀D^{v}₀T₀Q₀ (where R¹=Me and R³=—CH₂CH₂) siloxane, and also 20 g of Solvesso 150 in a multi-necked flask with blade stirrer with precision glass gland, reflux condenser, inert gas supply and thermometer, and the mixture was heated to a reaction temperature of from 80 to 90° C. in an inert gas atmosphere. Once the reaction temperature had been reached, the hydrosilylation reaction was initiated by addition of 10 ppm of platinum in the form of a Karstedt catalyst to the reaction mixture which until then was cloudy. The exothermic reaction here was controlled so as to avoid exceeding a reaction temperature of 90° C. After 4.5 hours it was not possible to detect any residual free SiH, and conversion was complete. The molar mass of the yellowish product according to GPC analysis (TI-IF) was Mₙ=4849 g/mol and M_{w}=78619 (M_{w}/Mₙ=16.21) and its viscosity was 8.9 Pa s. The Si content of the resultant product was ˜10% by weight, based on the pure substance, i.e. without solvent.

### S5

16.1 g of a siloxane of the formula M₁M^{H}₁D₁₂₃D^{H}₂₅T₀Q₀ (where R¹=Me) were intimately mixed with 63.2 g of allylpolyether 1, 2.6 g of an M₀M^{v}₆M^{H}₆D₁₇₃D^{H}₀D^{v}₀T₀Q₅ siloxane (where R¹=Me and R³=—CH₂CH₂, and also 20 g of Solvesso 150 in a multi-necked flask with stirrer with precision glass gland, reflux condenser, inert gas supply and thermometer, and the mixture was heated in an inert gas atmosphere to a reaction temperature of 80° C. Once the reaction temperature had been reached, the hydrosilylation reaction was initiated by addition of 10 ppm of platinum in the form of a Karstedt catalyst to the reaction mixture, which until then was cloudy. The exothermic reaction here was controlled so as to avoid exceeding a reaction temperature of 90° C. After 3 hours it was not possible to detect any residual free Sift and conversion was complete. The molar mass of the yellowish product according to GPC analysis was Mₙ=3886 g/mol and M_{w}=414335 g/mol (M_{w}/Mₙ=106.63) and its viscosity was 13 Pa s. The Si content of the resultant product was ˜8%, based on the pure substance, i.e. without solvent.

### Example 2

### Usage Example of a Composition According to Example 1 as Antifoam

The compositions according to the invention were tested in the ASTM D892 foam test. ASTM D892 describes the experimental set-up and the method for determining the foaming behaviour of oils. In this test, 190 ml of the test oil were placed in a 1 l measuring cylinder and heated to test temperature. At a given test temperature, a stream of air (humidity 65%) was introduced (1013 mbar) through a diffuser into the test liquid, see FIG. 1, where Y indicates the resultant foam. After 300 s the air supply was shut down, and the foam volume (FV) in millilitres between air layer and oil layer was measured. The foam collapse time (FC) in seconds was also measured, and ends when the entire surface of the liquid becomes visible. This test is usually carried out in accordance with ASTM D892 for lubricating oils at room temperature and at 93.5° C., with an airflow rate of 94 ml/min. It was modified to be appropriate to the present task by increasing the test temperature to 150° C. in a second pass, while simultaneously increasing the airflow rate to 1000 ml/min; the first pass was carried out at a temperature of 24° C. and an airflow rate of 94 ml/min.

The compositions according to the invention were compared with the organo-modified siloxanes that are prior art for transmission-system oils, and also with the polydimethylsiloxanes (silicone oils) with a dynamic viscosity of 300 000 cSt and 2 000 000 cSt which are used as prior art in engine oils and distillation processes and in cracking processes, and also with mixtures of the said silicone oils with silicone resins. According to the prior art it is advantageous to add the antifoam from a solvent. For the selected test, the antifoams were dissolved at 5% by weight in a solvent comprising aromatics (Hydrosol A 200 ND), and added to the oil requiring defoaming. A comparative experiment without any antifoam present was used as a blind sample. A conventional paraffin oil without other additives was used as hydrocarbon, with viscosity at 100° C. of 5.5 mm²/s. This oil is obtainable from Shell as SN 150 Generic oil.

Test Conditions
Test apparatus: ASTM D892
Test oil: SN 150 from Shell: Group 1 Generic oil with viscosity 5.5 mm²/s at 100° C.
Antifoam: 20 ppm of antifoam in the form of 5% by weight solution of the antifoam in Hydrosol 200 ND.
Comparative antifoam (not according to the invention):
   comp1: Silicone oil DC 200 Fluid, 300 000 cSt, from Dow Corning
   comp2: Silicone oil DC 200 Fluid, 2 000 000 cSt, from Dow Corning
   comp3: Tego Antifoam 2057, conventional commercially available antifoam for hydrocarbons based on organo-modified siloxanes from Evonik
   comp4: Tegopren 5851, conventional commercially available antifoam for hydrocarbons based on polydimethylpolyethersiloxane from Evonik
   comp5: Tegosivin HL 100, mixture of a branched silicone resin from Evonik, 5% by weight dissolved in silicone oil DC 200 Fluid, 300000 cSt
Test oil SN 150 without additions was used as blind sample B1. The test oil with addition of 1% by volume of Hydrosol A 200 ND was used as second blind sample B2.

TABLE 1
Foam volume (FV) and foam collapse time (FC) at
the stated temperatures according to example 2

| | FV [ml], | FC [s], | FV [ml], | FC [s], |
|---|---|---|---|---|
| Example | 150° C. | 150° C. | 24° C. | 24° C. |
| B1 | 800 | 43 | 420 | 280 |
| B2 | 850 | 44 | 400 | 270 |
| comp1 | 420 | 24 | 10 | 5 |
| comp2 | 300 | 18 | 10 | 5 |
| comp3 | 450 | 30 | 10 | 5 |
| comp4 | 600 | 35 | 10 | 5 |
| comp5 | 250 | 15 | 10 | 5 |
| S3 | 200 | 10 | 10 | 1 |
| S4 | 300 | 10 | 20 | 1 |
| S5 | 250 | 14 | 20 | 1 |

The results show the superiority of the compositions claimed according to the use when comparison is made with the prior art. In the experiments at ambient temperature, the compositions according to the use advantageously feature a low foam collapse time. In the experiments at 150° C., the compositions according to the use feature a markedly smaller foam volume when comparison is made with the conventional organo-modified polysiloxanes (comp3 and comp4) (at least 30% by volume reduction in foam volume, based on the prior art), and the compositions according to the use moreover feature markedly reduced foam collapse time.

The compounds claimed according to the use comprise only about 8-10% Si, in contrast to the comparative compounds.

### Example 2

### Usage Example in the Form of Antifoam in Processes above 500° C.

A glass coker apparatus can be used to gain understanding of foam formation and defoaming under coking conditions, these being the conditions for cracking processes subject to high temperatures in what is known as a delayed coker (“Foam Model for the Delayed Coker Pilot Unit”, Pradipta Chattopadhyay, The University of Tulsa, Dissertation 2006).

The system for simulating the coking processes is a 250 ml distillation apparatus with side arm (water-cooled) and a vessel to collect the decomposition products. The heating jacket, which encloses about half of the glass flask, can be equipment used by Aldrich (Glas-Col Series STM heating mantle), which can produce a temperature of up to 650° C. Once the liquid requiring defoaming has been added, 1 g of boiling chips is added. The closure system of the distillation apparatus has two apertures, one for temperature control by means of digital thermometer, while the other aperture permits use of a syringe which meters the antifoam solution to the system.

During the heating process, a foam is formed by boiling or decomposition processes at different temperatures depending on the test fluid. The antifoam solution is added when foam starts to form. The effectiveness of foam suppression is studied by observing and recording the time that elapses before more foam forms.

While this invention has been described in conjunction with the specific embodiments outlined above, it is evident that many alternatives, modifications, and variations will be apparent to those skilled in the art. Accordingly, the preferred embodiments of the invention as set forth above are intended to be illustrative, not limiting. Various changes may be made without departing from the spirit and scope of the inventions as defined in the following claims.

1. A method for defoaming a hydrocarbon that is liquid under conditions of usage, comprising:
utilizing a composition to defoam a hydrocarbon that is liquid under conditions of usage;
wherein the composition comprises:
a component A; and
a component D;
wherein A comprises a polymer obtained through a hydrosilylation reaction of compounds of the formula (I) with another compound of the formula (I) and/or with compounds C which have a C-C multiple bond and do not correspond to formula (I);
wherein the formula (I) is:
MₐM^{v}_{b}M^{H}_{c}D_{d}D^{H}ₑD^{v}_{f}T_{g}Qₕ formula (I);
where:
M=[R¹₃SiO_{1/2}];
M^{V}=[R³R¹₂SiO_{1/2}];
M^{H}=[R¹₂SiHO_{1/2}];
D=[R¹₂SiO_{2/2}];
D^{H}=[R¹SiHO_{2/2}];
D^{V}=[R³R¹SiO_{2/2}];
T=[R¹SiO_{3/2}];
Q=[SiO_{4/2}];
a=from 0 to 42;
b=from 0 to 42;
c=from 0 to 42;
d=from 5 to 600;
e=from 0 to 50;
f=from 0 to 50;
g=from 0 to 20; and
h=from 0 to 20;
with the proviso that the following conditions are satisfied:
a+b+c is greater than or equal to 2;
b+f is greater than 0;
c+e is greater than 0;
and 0.24*(a+b+c+d+e+f+g) is greater than (c+e);
where:
R¹ are mutually independently identical or different alkyl radicals having from 1 to 30 carbon atoms, or identical or different aryl radicals having from 6 to 30 carbon atoms or —OH or —OR²,
R² are mutually independently identical or different alkyl radicals having from 1 to 12 carbon atoms, or identical or different aryl radicals having from 6 to 12 carbon atoms,
R³ are mutually independently identical or different organic radicals having a terminal C-C double bond or having a terminal or internal C-C triple bond,
wherein component D is platinum-group-metal atoms and/or platinum-group-metal ions, preferably platinum atoms, rhodium atoms and ruthenium atoms, in particular platinum atoms.

2. The method according to claim 1;
wherein component A of the composition is a polymer obtained through a hydrosilylation reaction of compounds of the formula (I) and compounds of the formula (II); and
wherein the formula (II) is:
MᵢM^{H}ⱼDₖD^{H}ₗTₘQₙ formula (II);
where:
i=from 0 to 34;
j=from 0 to 34;
k=from 5 to 600;
l=from 0 to 50;
m=from 0 to 16;
n=from 0 to 16;
i+j is greater than or equal to 2; and
j+1 is greater than or equal to 2.

3. The method according to claim 1;
wherein component A of the composition comprises at least one polymer selected from the group consisting of:
a polymer obtained through a hydrosilylation reaction of compounds of the formula (I) with one or more unsaturated compounds C; and
a polymer obtained through a hydrosilylation reaction of compounds of the formula (I) with a compound of the formula (II) and with one or more unsaturated compounds C;
wherein the formula (II) is:
MᵢM^{H}ⱼDₖD^{H}ₗTₘQₙ formula (II);
where:
i=from 0 to 34;
j=from 0 to 34;
k=from 5 to 600;
l=from 0 to 50;
m=from 0 to 16;
n=from 0 to 16;
i+j is greater than or equal to 2; and
j+l is greater than or equal to 2.

4. The method according to claim 1;
wherein the composition further comprises a component B obtained through a hydrosilylation reaction of compounds of the formula (II) and unsaturated compounds C and/or that the composition used comprises one or more compounds C; and
wherein the formula (II) is:
MᵢM^{H}ⱼDₖD^{H}ₗTₘQₙ formula (II);
where:
i=from 0 to 34;
j=from 0 to 34;
k=from 5 to 600;
l=from 0 to 50;
m=from 0 to 16;
n=from 0 to 16;
i+j is greater than or equal to 2; and
j+l is greater than or equal to 2.

5. The method according to claim 1;
wherein compounds C of the composition are polyethers of the formula (III):
CH₂═CHCH₂O(C₂H₄O)ₒ(C₂H₃(CH₃)O)ₚ(C₂H₃(C₂H₅)O)_{q}(C₂H₃(Ph)O)ᵣR⁴ formula (III);
where:
R⁴ are mutually independently identical or different organic radicals which do not contain any multiple bond accessible to hydrosilylation;
o=from 0 to 200;
p from 0 to 200;
q=0 or from greater than 0 to 100;
r=0 or from greater than 0 to 100; and
when o+p+q+r is greater than 3, o is greater than 0.

6. The method according to claim 1;
wherein the silicon content of the composition is less than 25% by weight, based on the entire composition.

7. The method according to claim 1;
wherein the composition comprises further additions.

8. The method according to claim 1;
wherein, in distillation processes and vacuum distillation processes, the composition is added to a bottom product of the distillation process.

9. The method according to claim 1;
wherein the defoaming is achieved in processes at temperatures above 100° C.

10. The method according to claim 1;
wherein the composition is sprayed directly onto a foam in cracking processes.
